(19) United States
(12) Patent Application Publication
(10) Pub. No.: US 2008/0014218 A1
(43) Pub. Date: Jan. 17, 2008

(54) USE OF TIGHT JUNCTION AGONISTS TO FACILITATE PULMONARY DELIVERY OF THERAPEUTIC AGENTS

(75) Inventors: Alessio Fasano, West Friendship, MD (US); Blake Paterson, Baltimore, MD (US)

Correspondence Address: Connolly Bove Lodge Hutz, LLP;(FOR ALBA THERAPEUTICS), P.O. BOX 2207, WILMINGTON, DE, 19899-2207, US

(73) Assignee: ALBA THERAPEUTICS CORP., Baltimore, MD (US)

(21) Appl. No.: 11/737,264

(22) Filed: Apr. 19, 2007

Related U.S. Application Data
(60) Provisional application No. 60/792,973, filed on Apr. 19, 2006.

(51) Int. Cl. A61K 39/00; A61K 38/00; A61K 39/02; A61K 39/07; A61K 39/10; A61K 39/165; A61K 39/275; A61P 3/10; A61K 39/20; A61K 39/145; A61K 39/08; A61K 39/05; A61K 38/08

(52) U.S. Cl. 424/209.1; 424/184.1; 424/212.1; 424/219.1; 424/232.1; 424/245.1; 424/246.1; 424/247.1; 424/254.1; 424/256.1; 514/15; 514/16; 514/17; 514/2

(57) ABSTRACT

The present invention provides materials and methods to facilitate the pulmonary delivery of therapeutic agents. In some embodiments, agonists of tight junctions (e.g., zonulin agonists) are used in compositions to facilitate the uptake of therapeutic agents from the pulmonary mucosa.

### CROSS REFERENCE TO RELATED APPLICATIONS

This application claims priority to U.S. provisional patent application Ser. No. 60/792,973, filed Apr. 19, 2006, the contents of which are specifically incorporated herein by reference.

### FIELD OF THE INVENTION

The present invention provides materials and methods to facilitate the pulmonary delivery of therapeutic agents. In some embodiments, agonists of biological pathways responsible for opening and closing tight junctions (e.g., tight junction agonists, zonulin agonists, ZOT agonists) are used in compositions to facilitate the uptake of therapeutic agents from the pulmonary mucosa.

### BACKGROUND OF THE INVENTION

Pulmonary delivery of therapeutic agents has been used to treat various conditions in humans. Typically, pulmonary delivery compositions are designed to be delivered to the subject in need of the therapeutic agent by inhalation so that the therapeutic agent is delivered to the lung. Pulmonary delivery of a therapeutic agent can be accomplished by a variety of techniques, for example, by using liquid nebulizers, aerosol-based metered dose inhalers (MDI's), and/or dry powder dispersion devices. Pulmonary delivery can be effective both for systemic delivery and for localized delivery to treat diseases of the lungs.

Some therapeutic agents delivered to the lung are readily absorbed through the alveolar region directly into blood circulation. Others, particularly macromolecules (e.g., proteins, polypeptides and nucleic acids), are less well absorbed. Numerous efforts to improve the uptake of therapeutic agents delivered to the pulmonary mucosa have been made. For example, United States Pat. RE37,053 relates to particles incorporating surfactants for pulmonary drug delivery, U.S. Pat. No. 6,932,962 relates to aerosol drug formulations containing hydrofluoroalkanes and alkyl saccharides, and U.S. Pat. No. 5,635,161 relates to aerosol drug formulations containing vegetable oils. Notwithstanding these efforts, there remains a need in the art for methods and compositions to improve the uptake of therapeutic agents from the pulmonary mucosa. This need and others are met by the present invention.

### SUMMARY OF THE INVENTION

In one embodiment, the present invention provides pulmonary dosage compositions. Such compositions may comprise one or more therapeutic agents and a pulmonary absorption enhancing amount of one or more tight junction agonists. As used herein, a “tight junction agonist” is a compound that mediates or facilitates or augments the physiological, transient opening of tight junctions, for example, the tight junctions between adjacent epithelial cells. An example of a tight junction agonist is zonula occludens toxin (ZOT), which is produced by Vibrio cholerae. A ZOT receptor agonist is a compound which is believed to mediate tight junction opening through the same receptor utilized by ZOT. In another embodiment, a tight junction agonist may comprise zonulin. In some embodiments, a tight junction agonist may comprise a peptide. In some embodiments, a tight junction agonist may be a fragment of ZOT and/or zonulin. In some embodiments, a tight junction agonist comprising a peptide may comprise the amino acid sequence FCIGRL (SEQ ID NO: 1). A tight junction agonist comprising a peptide may comprise from about 6 to about 50 amino acids, from about 6 to about 25 amino acids, or from about 6 to about 10 amino acids.

A pulmonary dosage composition according to the invention may comprise one or more therapeutic agents. Examples of suitable therapeutic agents include, but are not limited to, antibiotics, anti-inflammatories, analgesics, insulin and vaccines. Therapeutic agents for use in the invention may be of any type known to those of skill in the art, for example, small molecules, peptides, proteins, lipids, carbohydrates, and combinations thereof.

Pulmonary dosage compositions of the invention may be liquids (e.g., aqueous solutions, emulsions, suspensions and the like). In some embodiments, a pulmonary dosage composition may be an aqueous solution, for example, a saline solution.

Pulmonary dosage compositions of the invention may also comprise one or more pharmaceutically acceptable excipients. Typical excipients that may be included in the compositions of the invention include, but are not limited to, sugars, salts, buffer salts, stabilizers, surfactants, preservatives and the like. Any pharmaceutically acceptable excipient known to those of skill in the art may be used.

An example of a pulmonary dosage composition of the invention is an aqueous solution comprising a tight junction agonist comprising a peptide comprising the sequence FCIGRL and also comprising at least one therapeutic agent selected from the group consisting of insulin, insulin modified by chemical or enzymatic means (including mutations introduced using recombinant DNA technology), parathyroid hormone, parathyroid hormone antagonist, calcitonin, vasopressin, renin, prolactin, growth hormone, thyroid stimulating hormone, corticotropin, corticotropin-releasing factor, follicle stimulating hormone, luteinizing hormone, chorionic gonadotropin, atrial peptides, interferon, tissue plasminogen activator, gammaglobulins, Factor VII, Factor VIII, growth hormone releasing hormone, luteinizing hormone releasing hormone, somatostatin and cholecystokinins.

The present invention also provides methods for treating animals (e.g., mammals including humans) by administering to a lung of the animal a composition comprising one or more therapeutic agents and a pulmonary absorption enhancing amount of one or more tight junction agonist. An example of a method of treating an animal is a method treating diabetes in an animal (e.g., a mammal such as a human) in need thereof, comprising administering to a lung of the animal a composition comprising insulin and/or an insulin derivative and a pulmonary absorption enhancing amount of one or more tight junction agonist. Compositions for use in methods of the invention may be liquids or aqueous solutions and may comprise one or more pharmaceutically acceptable excipients as described above.

The present invention also provides a method of inducing an immune response in an animal (e.g., a mammal such as a human), comprising administering to a lung of the animal a composition comprising one or more antigens and a pulmonary absorption enhancing amount of one or more tight junction agonists. Compositions for use in methods of inducing an immune response may further comprise one or more adjuvants (i.e., compounds that promote an enhanced immune response).

The present invention also provides immunogenic compositions. Such compositions may comprise one or more antigens and a pulmonary absorption enhancing amount of one or more tight junction agonists. Examples of antigens that may be included in immunogenic compositions of the invention include, but are not limited to, measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens. Such compositions may further comprise one or more adjuvants. Immunogenic compositions of the invention may be liquids and may comprise one or more pharmaceutically acceptable excipients as described above.

In another embodiment, the present invention provides compositions and methods for the pulmonary delivery of vaccines. Vaccines of the invention may be formulated for pulmonary delivery. Such vaccines may comprise one or more antigens and a pulmonary absorption enhancing amount of one or more tight junction agonists (e.g., a ZOT receptor agonist). Any antigen capable of inducing a protective immune response may be used in the vaccines of the invention. Examples of suitable antigens include, but are not limited to, measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens. Such vaccines may further comprise one or more adjuvants. Vaccines of the invention may be liquids and may comprise one or more pharmaceutically acceptable excipients as described above.

### DETAILED DESCRIPTION OF THE INVENTION

Definitions

As used herein, “a” or “an” may mean one or more. As used herein in the claim(s), when used in conjunction with the word “comprising”, the words “a” or “an” may mean one or more than one. As used herein “another” may mean at least a second or more.

As used herein, “adjuvant” refers to a compound that induces, enhances, and/or augments an immune response to an antigen.

As used herein, “antigen” refers to any compound that can elicit an immune response, for example, which can elicit production of an antibody that specifically binds to the antigen.

As used herein, “immunogenic composition” refers to any composition comprising an antigen.

As used herein, “vaccine” refers to an immunogenic composition capable of eliciting a protective immune response when administered to a subject. A protective immune response is one that reduces the severity of disease when a vaccinated subject is contacted with the disease causing agent (e.g., virus, bacterium, etc). Examples of a reduction in severity of a disease include, prevention of disease, delay in onset of disease, decreased severity of symptoms, decreased morbidity, and delayed mortality.

As used herein, a “tight junction agonist” is a compound that mediates or facilitates or augments the physiological, transient opening of tight junctions. Tight junctions are structures that form a barrier between adjacent epithelial cells (Johnson and Quay, Expert Opin Drug Deliv. March 2005; 2(2):281-98). An example of a tight junction agonist is zonula occludens toxin (ZOT), which is produced by Vibrio cholerae. A ZOT receptor agonist is a tight junction agonist which is believed to mediate tight junction opening through the same receptor utilized by ZOT. Tight junction agonists also include zonulin.

Tight Junction Agonists

Compositions of the invention typically comprise one or more tight junction agonists. A tight junction agonist facilitates absorption of a therapeutic agent. Further, the absorption occurs through the mucosa, and more particularly through the pulmonary mucosa. Thus, a tight junction agonist as used herein is a compound that mediates the physiological, transient opening of tight junctions. In some embodiments, a tight junction agonist may operate by binding to the ZOT receptor, i.e., may be a ZOT receptor agonist.

In some embodiments, a tight junction agonist may comprise a peptide comprising the amino acid sequence FCIGRL and/or functional derivatives of this sequence. Functional derivatives of peptide FCIGRL include, for example, Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met. In some embodiments, a tight junction agonist may consist of a peptide having the sequence FCIGRL and/or functional derivatives of this sequence as described herein.

Further, functional derivatives of peptide FCIGRL include: Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22). Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

When the tight junction agonist comprises a peptide, any length of peptide may be used. Generally, the size of the peptide agonist will range from about 6 to about 100, from about 6 to about 90, from about 6 to about 80, from about 6 to about 70, from about 6 to about 60, from about 6 to about 50, from about 6 to about 40, from about 6 to about 30, from about 6 to about 25, from about 6 to about 20, from about 6 to about 15, from about 6 to about 14, from about 6 to about 13, from about 6 to about 12, from about 6 to about 11, from about 6 to about 10, from about 6 to about 9, or from about 6 to about 8 amino acids in length. Peptide agonists of the invention may be from about 8 to about 100, from about 8 to about 90, from about 8 to about 80, from about 8 to about 70, from about 8 to about 60, from about 8 to about 50, from about 8 to about 40, from about 8 to about 30, from about 8 to about 25, from about 8 to about 20, from about 8 to about 15, from about 8 to about 14, from about 8 to about 13, from about 8 to about 12, from about 8 to about 11, or from about 8 to about 10 amino acids in length. Peptide agonists of the invention may be from about 10 to about 100, from about 10 to about 90, from about 10 to about 80, from about 10 to about 70, from about 10 to about 60, from about 10 to about 50, from about 10 to about 40, from about 10 to about 30, from about 10 to about 25, from about 10 to about 20, from about 10 to about 15, from about 10 to about 14, from about 10 to about 13, or from about 10 to about 12 amino acids in length. Peptide agonists of the invention may be from about 12 to about 100, from about 12 to about 90, from about 12 to about 80, from about 12 to about 70, from about 12 to about 60, from about 12 to about 50, from about 12 to about 40, from about 12 to about 30, from about 12 to about 25, from about 12 to about 20, from about 12 to about 15, or from about 12 to about 14 amino acids in length. Peptide agonists of the invention may be from about 15 to about 100, from about 15 to about 90, from about 15 to about 80, from about 15 to about 70, from about 15 to about 60, from about 15 to about 50, from about 15 to about 40, from about 15 to about 30, from about 15 to about 25, from about 15 to about 20, from about 15 to about 19, from about 15 to about 18, or from about 15 to about 17 amino acids in length. A tight junction agonist of the invention may comprise a peptide comprising about 6, about 7, about 8, about 9, about 10, about 11, about 12, about 13, about 14, about 15, about 20, about 30, about 40, about 50, about 60, about 70, about 80, about 90, or about 100 amino acids. In some embodiments of the invention, peptides do not encompass full length ZOT or zonulin.

Peptide agonists can be chemically synthesized and purified using well-known techniques, such as described in High Performance Liquid Chromatography of Peptides and Proteins: Separation Analysis and Conformation, Eds. Mant et al., C.R.C. Press (1991), and a peptide synthesizer, such as Symphony (Protein Technologies, Inc.); or by using recombinant DNA techniques, i.e., where the nucleotide sequence encoding the peptide is inserted in an appropriate expression vector, e.g., an E. coli or yeast expression vector, expressed in the respective host cell, and purified from the cells using well-known techniques.

Therapeutic Agents

Compositions of the invention typically comprise one or more therapeutic agents and/or immunogenic agents. Therapeutic agents that can be used in the compositions include agents that act on any organ of the body, such as heart, brain, intestine, or kidneys. Examples of suitable therapeutic agents include, but are not limited to, glucose metabolism agents (e.g., insulin), antibiotics, antineoplastics, antihypertensives, antiepileptics, central nervous system agents, and immune system suppressants.

The particular therapeutic and/or immunogenic agent used in the compositions of the invention can be any small molecule compound, biologically active peptide, vaccine, or any other moiety. In some embodiments, therapeutic agents for use in the invention may be those that, in the absence of a tight junction agonist, are not adequately absorbed into the bloodstream through the mucosa.

Examples of drug compounds which can be employed as therapeutic agents in the present invention include, but are not limited to, drugs which act on the cardiovascular system, drugs which act on the central nervous system, antineoplastic drugs and antibiotics. Examples of drugs which act on the cardiovascular system include, but are not limited to, antihypertensives, statins, adenosine, dobutamine, dopamine, epinephrine, norepinephrine, and phentolamine. Others as are known in the art can also be used.

Examples of drugs which act on the central nervous system include, but are not limited to, doxapram, alfentanil, dezocin, nalbuphine, buprenorphine, naloxone, ketorolac, midazolam, and propofol. Other examples include, but are not limited to, antipsychotics, antidepressents, antiepileptics, and drugs used to treat Alzheimers disease. Others as are known in the art can also be used.

Examples of antineopiastic drugs include, but are not limited to, cytarabine, mitomycin, doxorubicin, vincristine and vinblastine, carboplatin, cisplatin, oxaloplatin, vinorelbine, docetaxel, paclitaxel, taxane, 5-fluorouridine related drugs, xeloda, germcitabine, and anthracline. Additional examples include, but are not limited to, Erbitux, Herceptin®, Avastin™, and estrogen receptor antagonists and agonists. Others as are known in the art can also be used.

Examples of antibiotics include, but are not limited to, methicillin, mezlocillin, piperacillin, cetoxitin, cefonicid, cefinetazole and aztreonam. Others as are known in the art can also be used.

Any type of therapeutic and/or immunogenic agent can be used in the practice of the invention. Examples of specific types of agents include, but are not limited to, RNAi, treatment aptamers, antivirals (e.g., amantadine, rimantadine, zanamavir and oseltamivir), immune suppressants (e.g., cyclosporine A), HIV fusion inhibitors (e.g., enfuvirtide), and HIV protease inhibitors, (e.g., ritonavir, saquinavir, indinavir, amprenavir, nelfinavir, lopinavir, atazanavir, entricitabine, and fosamprenavir calcium).

Examples of biologically active peptides that may be used as therapeutic agents in the practice of the present invention include, but are not limited to, hormones, lymphokines, globulins, and albumins. Examples of hormones which can be employed in the present invention include: testosterone, nandrolene, menotropins, insulin and urofolltropin. Other examples of biologically active peptides include: insulin modified by chemical or enzymatic means (including mutations introduced using recombinant DNA technology), parathyroid hormone, parathyroid hormone antagonist, calcitonin, vasopressin, renin, prolactin, growth hormone, thyroid stimulating hormone, corticotropin, corticotropin-releasing factor, follicle stimulating hormone, luteinizing hormone, chorionic gonadotropin, atrial peptides, interferon, tissue plasminogen activator, gammaglobulins, Factor VII, Factor VIII, growth hormone releasing hormone, luteinizing hormone releasing hormone, somatostatin and cholecystokinins. Others as are known in the art can also be used. If the biologically active ingredient is insulin and/or an insulin derivative, the pulmonary dosage composition is useful for the treatment of diabetes.

Examples of lymphokines which can be employed in the present invention include interferon-α, interferon-β, interferon-γ, interleukin-1, interleukin-2, interleukin-4 and interleukin-8.

Examples of globulins include α-globulins, β-globulins and γ-globulins (immunoglobulin). Examples of immunoglobulins which can be employed in the present invention include polyvalent IgG or specific IgG, IgA and IgM, e.g., anti-tetanus antibodies. An example of albumin which can be used is human serum albumin. Others as are known in the art can also be used.

Examples of antigens that can be used in the compositions of the invention (e.g., immunogenic and/or vaccine compositions) include peptides, proteins, microorganisms (e.g., attenuated and/or recombinant microorganisms), cells (e.g., cancer cells and/or recombinant cells) and viruses (e.g., attenuated and/or recombinant viruses). Examples of peptide antigens include the B subunit of the heat labile enterotoxin of enterotoxigenic E. coli, the B subunit of cholera toxin, capsular antigens of enteric pathogens, fimbriae or pili of enteric pathogens, HIV surface antigens, cancer antigens (e.g., cancer cells comprising antigens, isolated antigens, etc.), dust allergens, and acari allergens. Other immunogenic compounds as are known in the art can also be used.

Examples of attenuated microorganisms and viruses that can be used in the compositions of the invention (e.g., vaccine compositions) include those of enterotoxigenic Escherichia coli, enteropathogenic Escherichia coli, Vibrio cholerae, Shigella flexneri, Salmonella typhi and rotavirus (Fasano et al, In: Le Vaccinazioni in Pediatria, Eds. Vierucci et al, CSH, Milan, pages 109-121 (1991); Guandalini et al, In: Management of Digestive and Liver Disorders in Infants and Children, Elsevior, Eds. Butz et al, Amsterdam, Chapter 25 (1993); Levine et al, Sem. Ped. Infect. Dis., 5.243-250 (1994); and Kaper et al, Clin. Micrbiol. Rev., 8:48-86 (1995), each of which is incorporated by reference herein in its entirety).

Any antigen capable of inducing a protective immune response may be used in the vaccines of the invention. Examples of suitable antigens include, but are not limited to, measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

Formulations

Compositions of the invention may formulated for pulmonary delivery (e.g., may be pulmonary dosage forms). Typically such compositions may be provided as pharmaceutical aerosols, e.g., solution aerosols. Those of skill in the art are aware of many different methods and devices for the formation of pharmaceutical aerosols, for example, those disclosed by Sciarra and Sciarra, Aerosols, in Remington: The Science and Practice of Pharmacy, 20th Ed., Chapter 50, Gennaro et al. Eds., Lippincott, Williams and Wilkins Publishing Co., (2000).

Typically, compositions comprising a tight junction agonist (e.g., peptide agonist) comprise a pharmaceutically effective amount of the agonist. The pharmaceutically effective amount of agonist (e.g., peptide agonist) employed may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation.

In one embodiment, the dosage forms are in the form of a solution aerosol (i.e., comprise droplets). Typically, droplets will be about 10 microns or less in diameter. Droplets for use in the compositions of the invention may have a diameter of from about 0.1 microns to about 10 microns, from about 0.1 microns to about 9 microns, from about 0.1 microns to about 8 microns, from about 0.1 microns to about 7 microns, from about 0.1 microns to about 6 microns, from about 0.1 microns to about 5 microns, from about 0.1 microns to about 4 microns, from about 0.1 microns to about 3 microns, from about 0.1 microns to about 2 microns, from about 0.1 microns to about 1 micron, from about 0.1 microns to about 0.5 microns, from about 1 micron to about 10 microns, from about 1 micron to about 9 microns, from about 1 micron to about 8 microns, from about 1 micron to about 7 microns, from about 1 micron to about 6 microns, from about 1 micron to about 5 microns, from about 1 micron to about 4 microns, from about 1 micron to about 3 microns, from about 1 micron to about 2 microns, from about 2 microns to about 10 microns, from about 2 microns to about 9 microns, from about 2 microns to about 8 microns, from about 2 microns to about 7 microns, from about 2 microns to about 6 microns, from about 2 microns to about 5 microns, from about 2 microns to about 4 microns, or from about 2 microns to about 3 microns. In some embodiments, particles and/or droplets for use in the invention may be about 1 micron, about 2 microns, about 3 microns, about 4 microns, about 5 microns, about 6 microns, about 7 microns, about 8 microns, about 9 microns, or about 10 microns in diameter.

Compositions of the invention may comprise one or tight junction agonist at a level of from about 0.000001 wt % to about 50 wt %, from about 0.000001 wt % to about 45 wt %, from about 0.000001 wt % to about 40 wt %, from about 0.000001 wt % to about 35 wt %, from about 0.000001 wt % to about 30 wt %, from about 0.000001 wt % to about 25 wt %, from about 0.000001 wt % to about 20 wt %, from about 0.000001 wt % to about 15 wt %, from about 0.000001 wt % to about 10 wt %, from about 0.000001 wt % to about 5 wt %, from about 0.000001 wt % to about 2.5 wt %, from about 0.000001 wt % to about 1 wt %, from about 0.000001 wt % to about 0.1 wt %, from about 0.000001 wt % to about 0.01 wt %, from about 0.000001 wt % to about 0.001 wt %, from about 0.000001 wt % to about 0.0001 wt %, from about 0.000001 wt % to about 0.00005 wt %, from about 0.0001 wt % to about 50 wt %, from about 0.0001 wt % to about 45 wt %, from about 0.0001 wt % to about 40 wt %, from about 0.0001 wt % to about 35 wt %, from about 0.0001 wt % to about 30 wt %, from about 0.0001 wt % to about 25 wt %, from about 0.0001 wt % to about 20 wt %, from about 0.0001 wt % to about 15 wt %, from about 0.0001 wt % to about 10 wt %, from about 0.0001 wt % to about 5 wt %, from about 0.0001 wt % to about 2.5 wt %, from about 0.0001 wt % to about 1 wt %, from about 0.0001 wt % to about 0.1 wt %, from about 0.0001 wt % to about 0.01 wt %, from about 0.0001 wt % to about 0.001 wt %, from about 0.0001 wt % to about 0.0005 wt %, from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more tight junction agonists at a level of about 0.00001 wt %, about 0.00005 wt %, about 0.0001 wt %, about 0.0005 wt %, about 0.001 wt %, about 0.005 wt %, about 0.01 wt %, about 0.05 wt %, about 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or more therapeutic agents and/or immunogenic agents at a concentration sufficient to cause the desired biological response (e.g., at a pharmaceutically effective concentration). Compositions of the invention may comprise one or therapeutic and/or immunogenic agents at a level of from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more therapeutic and/or immunogenic agents at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

Compositions of the invention may comprise one or pharmaceutically acceptable excipients at a level of from about 0.1 wt % to about 50 wt %, from about 0.1 wt % to about 45 wt %, from about 0.1 wt % to about 40 wt %, from about 0.1 wt % to about 35 wt %, from about 0.1 wt % to about 30 wt %, from about 0.1 wt % to about 25 wt %, from about 0.1 wt % to about 20 wt %, from about 0.1 wt % to about 15 wt %, from about 0.1 wt % to about 10 wt %, from about 0.1 wt % to about 5 wt %, from about 0.1 wt % to about 2.5 wt %, from about 0.1 wt % to about 1 wt %, from about 0.1 wt % to about 0.5 wt %, from about 0.1 wt % to about 0.2 wt %, from about 1 wt % to about 50 wt %, from about 1 wt % to about 45 wt %, from about 1 wt % to about 40 wt %, from about 1 wt % to about 35 wt %, from about 1 wt % to about 30 wt %, from about 1 wt % to about 25 wt %, from about 1 wt % to about 20 wt %, from about 1 wt % to about 15 wt %, from about 1 wt % to about 10 wt %, from about 1 wt % to about 5 wt %, from about 1 wt % to about 2.5 wt %, from about 5 wt % to about 50 wt %, from about 5 wt % to about 45 wt %, from about 5 wt % to about 40 wt %, from about 5 wt % to about 35 wt %, from about 5 wt % to about 30 wt %, from about 5 wt % to about 25 wt %, from about 5 wt % to about 20 wt %, from about 5 wt % to about 15 wt %, from about 5 wt % to about 10 wt %, from about 5 wt % to about 9 wt %, from about 5 wt % to about 8 wt %, from about 5 wt % to about 7 wt %, or from about 5 wt % to about 6 wt % of the total weight of the composition. Compositions of the invention may comprise one or more pharmaceutically acceptable excipients at a level of about 0.1 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % based on the total weight of the composition.

A composition according to the present invention may be pre-mixed prior to administration, or can be formed in vivo when two or more components (e.g., a tight junction agonist and a therapeutic agent) are administered within 24 hours of each other. When administered separately, the components may be administered in either order (e.g. tight junction agonist first followed by therapeutic agent or therapeutic agent first followed by tight junction agonist). The components can be administered within a time span of about 12 hours, about 8 hours, about 4 hours, about 2 hours, about 1 hour, about 0.5 hour, about 0.25 hour, about 0.1 hour, about 1 minute, about 0.5 minute, or about 0.1 minute.

Methods of Use

The pharmaceutical compositions of the invention can be used for treating, ameliorating, and/or preventing a disease. Any disease may be treated using the compositions of the invention by selection of an appropriate therapeutic and/or immunogenic agent. In one embodiment, the present invention provides a method of treating diabetes by administering a composition comprising one or more tight junction agonist and one or more insulin and/or derivative thereof.

Examples of diseases that can be treated using the compositions of the invention include, but are not limited to, cancer, autoimmune diseases, vascular disease, bacterial infections, gastritis, gastric cancer, collagnenous colitis, inflammatory bowel disease, osteoporosis, systemic lupus erythematosus, food allergy, asthma, and irritable bowel syndrome. For example, to treat cancer of the colon or rectal area, a composition comprising a therapeutically effective amount of Erbitux (Cetuximab) and an absorption enhancing amount of one or more tight junction agonists may be administered to the lung of a patient in need thereof, to treat breast cancer, a composition comprising a therapeutically effective amount of Herceptin (Trastuzumab) and an absorption enhancing amount of one or more tight junction agonists may be administered to the lung of a patient in need thereof, and to treat various types of cancer, a composition comprising a therapeutically effective amount of Avastin (Bevacizumab) and an absorption enhancing amount of one or more tight junction agonist may be administered to the lung of a patient in need thereof. Further examples include treatment of osteoporosis using a composition comprising one or more tight junction agonists and a therapeutically effective amount of Fosamax (Alendronate) administered to the lung of a subject in need thereof, treatment of transplant rejection using a composition comprising one or more tight junction agonists and a therapeutically effective amount of Cyclosporin A administered to the lung of a subject in need thereof, treatment of anemia using a composition comprising one or more tight junction agonists and a therapeutically effective amount of erythropoietin administered to the lung of a subject in need thereof, and treatment of hemophilia using a composition comprising one or more tight junction agonists and a therapeutically effective amount of Factor VIII administered to the lung of a subject in need thereof.

The following examples are provided for illustrative purposes only, and are in no way intended to limit the scope of the present invention.

### EXAMPLE 1

The following experiments were performed with tight junction agonist peptide FCIGRL.

EpiAirway™ inserts, media and DPBS (Dulbecco Phosphate Buffered Saline) were provided by MatTek Corporation, Ashland, Mass. These tissues are derived from normal Type 2 human tracheal/bronchial epithelial cells and cultured on semi-permeable synthetic membranes to form a pseudostratified, differentiated cell layer resembling the epithelial tissue of the conducting human airways.

Upon receipt of the inserts, they were transferred from medium-supplemented agarose gels to six well plates containing pre-warmed media, provided by MatTek Corp., and incubated in a CO₂ incubator at 37° C. (5% CO₂ in air) for at least 48 hours to allow the tissues to equilibrate and secrete mucin. The media was changed every other day until the day of the study. EpiAirway™ cells continue to divide and differentiate over time, thus the number of days in culture may affect experimental results. All experiments were conducted within four days of receipt for each lot utilized.

ENDO-OHM™ chambers (World Precision Instruments) were used to measure transepithelial electrical resistance (TEER) across the EpiAirway™ inserts. Three ENDO-OHM™ chambers were placed on a slide warmer and connected to an EVOM™ Epithelial Voltohmmeter (World Precision Instruments) via a data switch box. The temperature of the slide warmer was set to insure that pre-warmed media in the ENDO-OHM™ chambers would remain at approximately 37° C. for at least one hour. All DPBS solutions for these studies were used at 37° C. Excess mucin produced by the EpiAirway™ cells was removed from the apical surface with a single wash of 500 μL of DPBS before placement in the ENDO-OHM™ chambers. After removing the mucin, DPBS (250 μL) was added to the apical side and the initial control TEER was taken. Then 250 μL of DPBS containing 2× the final concentration of tight junction agonist per insert was applied to the apical side; the solution was mixed with gentle pipetting. TEER values were measured and recorded at times described for each experiment below. For experiments where the reversal of tight junction agonist effects on TEER were examined, DPBS containing agonist was removed, inserts were washed once with 250 μL of DPBS and fresh DPBS (500 μL) added. Also, fresh pre-warmed media was added to the basolateral side of the inserts. Measurements were made over the next 30 min to monitor TEER.

Initial TEER readings of EpiAirway™ inserts used for these experiments averaged 269±71 ohm-cm². For comparisons, TEER readings were expressed as a percentage of the initial TEER value.

Apical to basolateral flux of 4 kDa-FITC labeled dextran was measured using a continuous flow system. Three separate syringe pumps were used to perfuse OPTIMEM I™ (low phenol red) media through individual chambers of a CULTEX™ apparatus (n=3 chambers per apparatus) at a flow rate of approximately 0.33 mL/min. The CULTEX™ apparatus (Vitrocell Systems) was maintained at 37° C. A 2 mg/mL solution (400 μL) of 4kDa FITC-dextran containing 0, 0.3, or 10 μg/mL tight junction agonist was placed on the apical side of MatTek inserts. Three minute fractions of the media eluting from each chamber were collected and analyzed for FITC-dextran fluorescence (485 nm excitation/528 nm emission) using a fluorescent plate reader.

Portions of the tight junction agonist were stored at −20° C. in the presence of a desiccant until needed. Each experiment used a fresh portion of agonist. Sufficient sterile cold DPBS was added to the agonist to make a 10 mg/mL stock solution. The stock solution was rapidly diluted into larger volumes of sterile DPBS to obtain the desired concentrations.

A dose of 1 μg/mL tight junction agonist induced a time dependent decrease in TEER as compared to the DPBS control. Six concentrations of agonist, ranging from 0 to 10 μg/mL, were applied to the apical side of EpiAirway™ inserts and TEER evaluated at 0, 1, 5, 10, 15, 20, and 30 min. The agonist induced a decrease in TEER at all concentrations tested with the maximal decrease in TEER being observed at the initial 1 minute time point for all concentrations. Unexpectedly, the greatest decline in TEER was noted at 0.1 and 0.3 μg/mL of agonist. After the 30 min TEER measurement, agonist-containing DPBS was removed and fresh DPBS added to determine the reversibility of the agonist's effect. In this phase of the experiment, simply removing agonist solutions and a single wash did not result in significant recovery of TEER during the period monitored (25 min), with the exception of the lowest concentration (0.01 μg/mL). The results from this study demonstrate that a tight junction agonist caused significant changes in TEER in the pulmonary epithelial cell model system at all concentrations tested. Based upon these data, two concentrations of agonist (0.3 and 10 μg/mL) were chosen for further testing in macromolecular transport (flux) experiments below.

The effect of two doses of tight junction agonist on 4 kDa FITC-dextran flux across EpiAirway™ cells in a continuous flow system was determined. Agonist increased 4 kDa FITC-dextran flux at the 0.3 μg/mL concentration but not at the 10 μg/mL concentration as compared to the DPBS control. In addition, in contrast to the TEER studies the increased flux of FITC-Dextran observed with 0.3 μg/ml of agonist appeared to return to levels similar to DPBS by 20 minutes. When this experiment was repeated, there was a trend of a dose dependent effect of agonist on FITC-dextran flux with a greater flux observed at the 10 μg/mL than at the 0.3 μg/mL dose. As in the first flux experiment, the flux of FITC-Dextran achieved with agonist appeared returned to levels comparable to DPBS after approximately 20 minutes.

### EXAMPLE 2

An aqueous saline solution containing 10 μg of salmon calcitonin was instilled into the lungs of rats and serum calcitonin levels were measured by ELISA. An aqueous saline solution containing 10 μg of salmon calcitonin and 1 mg of the tight junction agonist peptide FCIGRL was instilled into the lungs of rats and serum calcitonin levels were measured by ELISA. The presence of tight junction agonist resulted in an increase of approximately three-fold in observed peak serum calcitonin levels and an increase of approximately 10.6 fold in area under the curve (AUC).

While the invention has been described in detail, and with reference to specific embodiments thereof, it will be apparent to one of ordinary skill in the art that various changes and modifications can be made therein without departing from the spirit and scope thereof and such changes and modifications may be practiced within the scope of the appended claims. All patents and publications herein are incorporated by reference to the same extent as if each individual publication was specifically and individually indicated to be incorporated by reference in their entirety.

What is claimed is:

1. A pulmonary dosage composition, comprising:
one or more therapeutic agents; and
a pulmonary absorption enhancing amount of one or more tight junction agonists.

2. A composition according to claim 1, wherein at least one agonist comprises a peptide.

3. A composition according to claim 2, wherein the peptide comprises the sequence FCIGRL.

4. A composition according to claim 2, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

5. A composition according to claim 2, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 1), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

6. A composition according to claim 2, wherein the peptide comprises from about 6 to about 10 amino acids.

7. A composition according to claim 1, wherein at least one therapeutic agent is selected from the group consisting of antibiotics, anti-inflammatories, analgesics, insulin and vaccines.

8. A composition according to claim 1, wherein at least one therapeutic agent is selected from the group consisting of small molecules, peptides, proteins, lipids, carbohydrates, and combinations thereof.

9. A composition according to claim 1, wherein the composition is in aqueous solution.

10. A composition according to claim 1, wherein the composition is in a saline solution.

11. A composition according to claim 1, wherein the composition further comprises one or more pharmaceutically acceptable excipients.

12. A composition according to claim 1, wherein the tight junction agonist is a peptide comprising the sequence FCIGRL and the composition is in aqueous solution and the composition comprises one or more therapeutic agents selected from the group consisting of small molecules, peptides, proteins, lipids, and carbohydrates and combinations thereof.

13. A method of treating an animal, comprising:
administering to a lung of the animal a composition comprising one or more therapeutic agents and a pulmonary absorption enhancing amount of a tight junction agonist.

14. A method according to claim 13, wherein the animal is a mammal.

15. A method according to claim 13, wherein the animal is a human.

16. A method according to claim 13, wherein at least one agonist comprises a peptide.

17. A method according to claim 16, wherein the peptide comprises the sequence FCIGRL.

18. A method according to claim 16, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

19. A method according to claim 16, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

20. A method according to claim 16, wherein the peptide comprises from about 6 to about 10 amino acids.

21. A method according to claim 13, wherein at least one therapeutic agent is selected from the group consisting of antibiotics, anti-inflammatories, analgesics, insulin and vaccines.

22. A method according to claim 13, wherein at least one therapeutic agent is selected from the group consisting of small molecules, peptides, proteins, lipids, carbohydrates, and combinations thereof.

23. A method according to claim 13, wherein the composition is in aqueous solution.

24. A method according to claim 13, wherein the composition is in a saline solution.

25. A method according to claim 13, wherein the composition further comprises one or more pharmaceutically acceptable excipients.

26. A method according to claim 13, wherein the tight junction agonist is a peptide comprising the sequence FCIGRL and the composition is in aqueous solution and the composition comprises one or more therapeutic agents selected from the group consisting of small molecules, peptides, proteins, lipids, carbohydrates, and combinations thereof.

27. A method treating diabetes in an animal in need thereof, comprising:
administering to a lung of the animal a composition comprising insulin and/or a derivative thereof and a pulmonary absorption enhancing amount of a tight junction agonist.

28. A method according to claim 27, wherein the animal is a mammal.

29. A method according to claim 27, wherein the animal is a human.

30. A method according to claim 27, wherein at least one agonist comprises a peptide.

31. A method according to claim 30, wherein the peptide comprises the sequence FCIGRL.

32. A method according to claim 30, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

33. A method according to claim 30, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

34. A method according to claim 30, wherein the peptide comprises from about 6 to about 10 amino acids.

35. A method according to claim 27, wherein the composition is in aqueous solution.

36. A method according to claim 27, wherein the composition is in a saline solution.

37. A method according to claim 27, wherein the composition further comprises one or more pharmaceutically acceptable excipients.

38. A method according to claim 27, wherein the tight junction agonist is a peptide comprising the sequence FCIGRL and the composition is in aqueous solution and the composition comprises human insulin and/or a pharmaceutically acceptable derivative thereof.

39. A method of inducing an immune response in an animal, comprising:
administering to a lung of the animal a composition comprising one or more antigens and a pulmonary absorption enhancing amount of a tight junction agonist.

40. A method according to claim 39, further comprising administering an adjuvant.

41. A method according to claim 39, wherein the composition further comprises an adjuvant.

42. A method according to claim 39, wherein the animal is a mammal.

43. A method according to claim 39, wherein the animal is a human.

44. A method according to claim 39, wherein at least one agonist comprises a peptide.

45. A method according to claim 44, wherein the peptide comprises the sequence FCIGRL.

46. A method according to claim 44, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

47. A method according to claim 44, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

48. A method according to claim 44, wherein the peptide comprises from about 6 to about 10 amino acids.

49. A method according to claim 39, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

50. A method according to claim 39, wherein the composition is in aqueous solution.

51. A method according to claim 39, wherein the composition is in a saline solution.

52. A method according to claim 39, wherein the composition further comprises one or more pharmaceutically acceptable excipients.

53. A method according to claim 39, wherein the tight junction agonist is a peptide comprising the sequence FCIGRL and the composition is in aqueous solution and the composition comprises one or more antigens selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

54. An immunogenic composition, comprising:
one or more antigens and a pulmonary absorption enhancing amount of a tight junction agonist.

55. An immunogenic composition according to claim 54, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

56. A composition according to claim 54, wherein at least one agonist comprises a peptide.

57. A composition according to claim 56, wherein the peptide comprises the sequence FCIGRL.

58. A composition according to claim 57, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

59. A composition according to claim 57, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 1), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

60. A composition according to claim 57, wherein the peptide comprises from about 6 to about 10 amino acids.

61. A composition according to claim 54, wherein the composition is in aqueous solution.

62. A composition according to claim 54, wherein the composition is in a saline solution.

63. A composition according to claim 54, wherein the composition further comprises one or more pharmaceutically acceptable excipients.

64. A composition according to claim 54, wherein the tight junction agonist is a peptide comprising the sequence FCIGRL and the composition is in aqueous solution and the composition comprises at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

65. A vaccine comprising one or more antigens and a pulmonary absorption enhancing amount of a tight junction agonist.

66. A vaccine according to claim 65, wherein at least one antigen is selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.

67. A vaccine according to claim 65, wherein at least one agonist comprises a peptide.

68. A vaccine to claim 67, wherein the peptide comprises the sequence FCIGRL.

69. A vaccine according to claim 68, wherein the peptide comprises a sequence selected from the group consisting of Xaa₁ Cys Ile Gly Arg Leu (SEQ ID NO: 2), Phe Xaa₂ Ile Gly Arg Leu (SEQ ID NO: 3), Phe Cys Xaa₃ Gly Arg Leu (SEQ ID NO: 4), Phe Cys Ile Xaa₄ Arg Leu (SEQ ID NO: 5), Phe Cys Ile Gly Xaa₅ Leu (SEQ ID NO: 6), and Phe Cys Ile Gly Arg Xaa₆ (SEQ ID NO: 7), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

70. A vaccine according to claim 68, wherein the peptide comprises a sequence selected from the group consisting of Xaa₁ Xaa₂ Ile Gly Arg Leu (SEQ ID NO: 8), Xaa₁ Cys Xaa₃ Gly Arg Leu (SEQ ID NO: 9), Xaa₁ Cys Ile Xaa₄ Arg Leu (SEQ ID NO: 10), Xaa₁ Cys Ile Gly Xaa₅ Leu (SEQ ID NO: 11), Xaa₁ Cys Ile Gly Arg Xaa₆ (SEQ ID NO: 12), Phe Xaa₂ Xaa₃ Gly Arg Leu (SEQ ID NO: 13), Phe Xaa₂ Ile Xaa₄ Arg Leu (SEQ ID NO: 14), Phe Xaa₂ Ile Gly Xaa₅ Leu (SEQ ID NO: 15), Phe Xaa₂ Ile Gly Arg Xaa₆ (SEQ ID NO: 16), Phe Cys Xaa₃ Xaa₄ Arg Leu (SEQ ID NO: 17), Phe Cys Xaa₃ Gly Xaa₅ Leu (SEQ ID NO: 18), Phe Cys Xaa₃ Gly Arg Xaa₆ (SEQ ID NO: 19), Phe Cys Ile Xaa₄ Xaa₅ Leu (SEQ ID NO: 20), Phe Cys Ile Xaa₄ Arg Xaa₆ (SEQ ID NO: 21), and Phe Cys Ile Gly Xaa₅ Xaa₆ (SEQ ID NO: 22), wherein Xaa₁ is selected from the group consisting of Ala, Val, Leu, Ile, Pro, Trp, Tyr, and Met; Xaa₂ is selected from the group consisting of Gly, Ser, Thr, Tyr, Asn, and Gln; Xaa₃ is selected from the group consisting of Ala, Val, Leu, Ile, Pro, Trp, and Met; Xaa₄ is selected from the group consisting of Gly, Ser, Thr, Tyr, Asn, Ala, and Gln; Xaa₅ is selected from the group consisting of Lys and His; Xaa₆ is selected from the group consisting of Ala, Val, Leu, Ile, Pro, Trp, and Met.

71. A vaccine according to claim 68, wherein the peptide comprises from about 6 to about 15 amino acids.

72. A vaccine according to claim 65, wherein the vaccine is an aqueous solution.

73. A vaccine according to claim 65, wherein the vaccine is a saline solution.

74. A vaccine according to claim 65, wherein the vaccine further comprises one or more pharmaceutically acceptable excipients.

75. A vaccine according to claim 65, wherein the tight junction agonist is a peptide comprising the sequence FCIGRL and the vaccine is an aqueous solution and the vaccine comprises at least one antigen selected from the group consisting of measles virus antigens, mumps virus antigens, rubella virus antigens, Corynebacterium diphtheriae antigens, Bordetella pertussis antigens, Clostridium tetani antigens, Bacillus anthracis antigens, Haemophilus influenzae antigens, smallpox virus antigens, and influenza virus antigens.


SEQUENCE LISTING (22 sequences)

SEQ ID NO: 1 - protein, 6 aa, Artificial
  synthetic peptide
FCIGRL

SEQ ID NO: 2 - protein, 6 aa, Artificial
  synthetic peptide
  misc_feature (1)..(1): Xaa can be Ala, Val, Leu, Ile, Pro, Trp, Tyr, and Met
XCIGRL

SEQ ID NO: 3 - protein, 6 aa, Artificial
  synthetic peptide
  misc_feature (2)..(2): Xaa can be Gly, Ser, Thr, Tyr, Asn, and Gln
FXIGRL

SEQ ID NO: 4 - protein, 6 aa, Artificial
  synthetic peptide
  misc_feature (3)..(3): Xaa can be Ala, Val, Leu, Ile, Pro, Trp, and Met
FCXGRL

SEQ ID NO: 5 - protein, 6 aa, Artificial
  synthetic peptide
  misc_feature (4)..(4): Xaa can be Gly, Ser, Thr, Tyr, Asn, Ala, and Gln
FCIXRL

SEQ ID NO: 6 - protein, 6 aa, Artificial
  synthetic peptide
  misc_feature (5)..(5): Xaa can be Lys and His
FCIGXL

SEQ ID NO: 7 - protein, 6 aa, Artificial
  synthetic peptide
  misc_feature (6)..(6): Xaa can be Ala, Val, Leu, Ile, Pro, Trp, and Met
FCIGRX

SEQ ID NO: 8 - protein, 6 aa, Artificial
  synthetic peptide
  misc_feature (1)..(1): Xaa can be Ala, Val, Leu, Ile, Pro, Trp, Tyr, and Met
  misc_feature (2)..(2): Xaa can be Gly, Ser, Thr, Tyr, Asn, and Gln
XXIGRL

SEQ ID NO: 9 - protein, 6 aa, Artificial
  synthetic peptide
  misc_feature (1)..(1): Xaa can be Ala, Val, Leu, Ile, Pro, Trp, Tyr, and Met
  misc_feature (3)..(3): Xaa can be Ala, Val, Leu, Ile, Pro, Trp, and Met
XCXGRL

SEQ ID NO: 10 - protein, 6 aa, Artificial
  synthetic peptide
  misc_feature (1)..(1): Xaa can be Ala, Val, Leu, Ile, Pro, Trp, Tyr, and Met
  misc_feature (4)..(4): Xaa can be Gly, Ser, Thr, Tyr, Asn, Ala, and Gln
XCIXRL

SEQ ID NO: 11 - protein, 6 aa, Artificial
  synthetic peptide
  misc_feature (1)..(1): Xaa can be Ala, Val, Leu, Ile, Pro, Trp, Tyr, and Met
  misc_feature (5)..(5): Xaa can be Lys and His
XCIGXL

SEQ ID NO: 12 - protein, 6 aa, Artificial
  synthetic peptide
  misc_feature (1)..(1): Xaa can be Ala, Val, Leu, Ile, Pro, Trp, Tyr, and Met
  misc_feature (6)..(6): Xaa can be Ala, Val, Leu, Ile, Pro, Trp, and Met
XCIGRX

SEQ ID NO: 13 - protein, 6 aa, Artificial
  synthetic peptide
  misc_feature (2)..(2): Xaa can be Gly, Ser, Thr, Tyr, Asn, and Gln
  misc_feature (3)..(3): Xaa can be Ala, Val, Leu, Ile, Pro, Trp, and Met
FXXGRL

SEQ ID NO: 14 - protein, 6 aa, Artificial
  synthetic peptide
  misc_feature (2)..(2): Xaa can be Gly, Ser, Thr, Tyr, Asn, and Gln
  misc_feature (4)..(4): Xaa can be Gly, Ser, Thr, Tyr, Asn, Ala, and Gln
FXIXRL

SEQ ID NO: 15 - protein, 6 aa, Artificial
  synthetic peptide
  misc_feature (2)..(2): Xaa can be Gly, Ser, Thr, Tyr, Asn, and Gln
  misc_feature (5)..(5): Xaa can be Lys and His
FXIGXL

SEQ ID NO: 16 - protein, 6 aa, Artificial
  synthetic peptide
  misc_feature (2)..(2): Xaa can be Gly, Ser, Thr, Tyr, Asn, and Gln
  misc_feature (6)..(6): Xaa can be Ala, Val, Leu, Ile, Pro, Trp, and Met
FXIGRX

SEQ ID NO: 17 - protein, 6 aa, Artificial
  synthetic peptide
  misc_feature (3)..(3): Xaa can be Ala, Val, Leu, Ile, Pro, Trp, and Met
  misc_feature (4)..(4): Xaa can be Gly, Ser, Thr, Tyr, Asn, Ala, and Gln
FCXXRL

SEQ ID NO: 18 - protein, 6 aa, Artificial
  synthetic peptide
  misc_feature (3)..(3): Xaa can be Ala, Val, Leu, Ile, Pro, Trp, and Met
  misc_feature (5)..(5): Xaa can be Lys and His
FCXGXL

SEQ ID NO: 19 - protein, 6 aa, Artificial
  synthetic peptide
  misc_feature (3)..(3): Xaa can be Ala, Val, Leu, Ile, Pro, Trp, and Met
  misc_feature (6)..(6): Xaa can be Ala, Val, Leu, Ile, Pro, Trp, and Met
FCXGRX

SEQ ID NO: 20 - protein, 6 aa, Artificial
  synthetic peptide
  misc_feature (4)..(4): Xaa can be Gly, Ser, Thr, Tyr, Asn, Ala, and Gln
  misc_feature (5)..(5): Xaa can be Lys and His
FCIXXL

SEQ ID NO: 21 - protein, 6 aa, Artificial
  synthetic peptide
  misc_feature (4)..(4): Xaa can be Gly, Ser, Thr, Tyr, Asn, Ala, and Gln
  misc_feature (6)..(6): Xaa can be Ala, Val, Leu, Ile, Pro, Trp, and Met
FCIXRX

SEQ ID NO: 22 - protein, 6 aa, Artificial
  synthetic peptide
  misc_feature (5)..(5): Xaa can be Lys and His
  misc_feature (6)..(6): Xaa can be Ala, Val, Leu, Ile, Pro, Trp, and Met
FCIGXX